(19) United States
(12) Patent Application Publication
(10) Pub. No.: US 2022/0211637 A1
(43) Pub. Date: Jul. 7, 2022

(54) RANDOM COPOLYMER STABILIZED NANOPARTICLES ENCAPSULATING SOLUBLE HYDROPHILIC COMPOUNDS

(71) Applicant: The Trustees of Princeton University, Princeton, NJ (US)

(72) Inventors: Robert F. Pagels, Princeton, NJ (US); Chester E. Markwalter, Princeton, NJ (US); Justin Gourary, New York, NY (US); Robert K. Prud'homme, Lawrenceville, NJ (US)

(73) Assignee: The Trustees of Princeton University, Princeton, NJ (US)

(21) Appl. No.: 17/609,887

(22) PCT Filed: May 6, 2020
(86) PCT No.: PCT/US2020/031579

§ 371 Date: Nov. 9, 2021

Related U.S. Application Data
(60) Provisional application No. 62/845,613, filed on May 9, 2019.

(51) Int. Cl. A61K 9/51; B01J 13/08; A61K 31/711; A61K 31/7105; A61K 38/47; A61K 31/7036; A61K 38/14; A61K 9/16; A61K 38/12

(52) U.S. Cl. CPC A61K 9/5192; B01J 13/08; A61K 31/711; A61K 31/7105; A61K 38/12; A61K 31/7036; A61K 38/14; A61K 9/1652; A61K 38/47

(57) ABSTRACT

Disclosed is a process to make nanoparticles highly loaded with water soluble actives, including biologics such as proteins and peptides, which are stabilized by random copolymers. The random copolymers used have all been approved by the FDA for oral formulations. The nanoparticles have a hydrophilic core and a hydrophobic corona and can be further processed through a number of different routes. The process to make these particles is highly scalable and could be used industrially.

### CROSS-REFERENCE TO RELATED APPLICATIONS

This application claims priority to U.S. Provisional Application No. 62/845,613 filed May 9, 2019, which is hereby incorporated in its entirety by reference.

### STATEMENT REGARDING FEDERALLY SPONSORED RESEARCH OR DEVELOPMENT

This invention was made with government support under Contract IIP-1843551 awarded by the National Science Foundation. The government has certain rights in the invention

### TECHNICAL FIELD

The present disclosure relates to the process of forming nanoparticles having a hydrophilic core which are stabilized by random copolymers, formulating said nanoparticles into microparticles, and the compositions thereof.

### BACKGROUND

Oral delivery of water-soluble therapeutics, particularly biologics, requires complex drug delivery formulations. Biologics, including proteins, peptides, and nucleic acids such as DNA and RNA, have minimal oral bioavailability. These complex soluble molecules can degrade due to exposure to enzymes or extreme pH conditions. Special formulations, capable of protecting biologics from degradation during oral delivery, are required. These formulations can include nanoparticles or microparticles.

Random copolymers, including cellulosic-based polymers and poly(meth)acrylate-based polymers, have been developed specifically for oral delivery of therapeutics. The polymers impart specific characteristics to oral formulations including pH-responsiveness (enteric coatings), sustained release, and taste and odor masking. The polymers can be employed as coatings or as matrices.

Recently, a method to make nanoparticles comprising of a hydrophobic core stabilized by random copolymers, specifically cellulose-based polymers, has been described (WO/2019/055539). In that method, a hydrophobic active agent and stabilizing copolymer are dissolved in a non-polar solvent stream which is rapidly mixed with an aqueous non-solvent stream in a process called Flash NanoPrecipitation (FNP). Upon mixing, the hydrophobic active precipitates, forming the nanoparticle core. The hydrophobic groups of the stabilizing polymer stick to the nanoparticle surface, and the hydrophilic groups of the stabilizing polymer face the external aqueous phase. The FNP process is suitable for encapsulating agents that are poorly water soluble. However, FNP is not suitable for encapsulating water-soluble agents, including biologics, which will not precipitate in the aqueous non-solvent stream.

World Patents WO 2017/112828 A1 and WO 2015/200054, incorporated in their entirety herein, presented methods for encapsulating hydrophilic compounds in nanoparticles and microparticles. That method was termed “inverse Flash NanoPrecipitation” (iFNP). In the iFNP process, a hydrophilic active agent such as a biologic and stabilizing block copolymer are dissolved in a polar solvent stream which is rapidly mixed with a non-polar non-solvent stream. Upon mixing, the hydrophilic active precipitates, forming the nanoparticle core. The hydrophilic block(s) of the stabilizing polymer stick to the nanoparticle surface, and the hydrophobic block(s) of the stabilizing polymer face the external aqueous phase.

Until now, only well-defined block copolymers have been used as stabilizers.

### BRIEF SUMMARY

Aspects of the present disclosure are drawn to methods and systems utilizing random copolymers as stabilizers in the iFNP process, including those random copolymers used in oral drug delivery formulations.

In this disclosure, nanoparticles are described comprising of a hydrophilic core that are stabilized by random copolymers. These nanoparticles can range in size from about 10 nm to about 5000 nm. In certain embodiments, the hydrophilic core can include water-soluble small molecules, proteins, peptides, nucleic acids including DNA and RNA, and/or polysaccharides (also referred as a hydrophilic “agent” or “active” or “therapeutic” or “biologic”). The stabilizing random copolymer is composed of both polar (hydrophilic) and non-polar (hydrophobic) groups or monomers. The hydrophilic groups of the random copolymer anchor the polymer to the hydrophilic nanoparticle core. The hydrophobic groups of the random copolymer face outwards and provide steric stability. In certain embodiments, the stabilizing random copolymer can be a cellulosic polymer. For example, this stabilizing random copolymer can be hydroxypropyl cellulose, methyl cellulose, ethyl methyl cellulose, hydroxypropylmethylcellulose, carboxymethyl cellulose, or a combination of these. The cellulosic polymer can include hydroxypropyl, hydroxyethyl, hydroxymethyl, succinate, and/or acetate substitution(s). In another embodiment, poly(meth)acrylate-based random copolymers are used as stabilizers. Eudragit polymers produced by Evonik Industries are one commercialized version of these. However, the present disclosure is not limited to the aforementioned random copolymers.

In this disclosure, a method is described for making nanoparticles comprising of a hydrophilic core that are stabilized by random copolymers. In a process (method) according to the disclosure, a random copolymer is dissolved in a more polar solvent to form a process solution. For example, the polar process solvent can be, but are not limited to, water, dimethyl sulfoxide (DMSO), dimethylformamide (DMF), methanol, ethanol, N-methyl-2-pyrrolidone (NMP), or mixtures thereof. In an iteration of this disclosure, a hydrophilic agent or active, such as a biologic, is also included in the process solution with the random copolymer. In another iteration, a hydrophilic agent or active, such as a biologic, is also included in a separate polar process solution which may or may not be composed of the same solvent as the copolymer process solution. The water soluble active can have a solubility in water of greater than 0.01 mg/mL and/or a log P value of less than 3. The water soluble active can have a solubility in water of greater than 0.1 mg/mL and/or a log P value of less than 2. The water soluble active can have a solubility in water of greater than 1 mg/mL and/or a log P value of less than 1. In general, the solubility of the active in water is less important to the processing than the solubility of the active in the non-process solvent. It is important that the active has low solubility in the non-polar non-process solvent. For example, the active can have a solubility in the non-process solvent of less than 1 mg/mL, less than 0.1 mg/mL, and/or less than 0.01 mg/mL. Examples of water-soluble actives includes proteins (e.g., lysozyme and ovalbumin), polypeptides, linear polypeptides (e.g., proteins), cyclic polypeptides (e.g., vancomycin), branched polypeptides, glycosylated peptides (e.g., vancomycin), and other biologics and nonbiologic molecules. For example, the water soluble active can have a molecular weight of from about 100 Da, 200 Da, 500 Da, 1000 Da, 2000 Da, 5000 Da, 10000 Da, 20000 Da, and 40000 Da to about 1000 Da, 2000 Da, 5000 Da, 10000 Da, 20000 Da, 40000 Da, 100 kDa, 200 kDa, 500 kDa, and 1000 kDa. The process solution is mixed with a more nonpolar solvent (non-process stream). For example, the non-polar non-process solvent can be, but are not limited to, chloroform, dichloromethane (DCM), tetrahydrofuran (THF), acetone, or mixtures thereof. In some embodiments, there can be more than one non-process streams. Upon mixing the process and non-process stream, the hydrophilic agent rapidly precipitates, becoming the core of the nanoparticle. The hydrophilic groups within the stabilizing random copolymer precipitate onto the nanoparticle core and the hydrophobic groups within the stabilizing random copolymer form a shell.

In an embodiment, the process and non-process solutions are rapidly mixed in a continuous process. For example, the process solution(s) can be in a process stream(s), the non-process solvent(s) can be in a non-process solvent stream(s). The process stream can be continuously combined with the non-process stream in a confined mixing volume, and/or the formed nanoparticle can exit the confined mixing volume in an exit stream. For example, the process and non-process streams can be continuously combined in a Confined Impinging Jet Mixer or a Multi-Inlet Vortex Mixer.

In an embodiment of the disclosed method, the nonprocess solvent can be chloroform, dichloromethane, an alkane, hexane, an ether, diethyl ether, tetrahydrofuran (THF), toluene, acetone, or combinations. For example, the nonprocess solvent can be chloroform, dichloromethane, acetone, or combinations. For example, the polar process solvent and the nonprocess solvent can be miscible. In an embodiment, the process and nonprocess solvents are completely miscible at the volume ratios used in the nanoparticle formation process. Alternatively, in another embodiment, the process solvent is substantially soluble in the nonprocess solvent, where substantially soluble is defined as having 80% by volume of the process solvent miscible in the nonprocess solvent under volume ratios used in the nanoparticle formation process.

In an embodiment of the disclosed method, a time of mixing of the process solution with the nonprocess solvent is less than an assembly time of the nanoparticle. The hydrophilic agent and the copolymer can have a supersaturation level in the solution ranging from 10 to 10,000.

A method of the invention includes stabilizing the nanoparticle core through crosslinking of the copolymer. For example, the nanoparticle can be crosslinked during assembly. The crosslinking can occur after assembly.

In an embodiment of the disclosed method, the nanoparticles may be processed to form microparticles. In an embodiment, the microparticles can be formed in an emulsion process. In another embodiment, the microparticles can be formed by spray drying. A polymer of the same or different type as the nanoparticle stabilizer can be included to impart desired functionality. The obtained powders may be processed or formulated according to techniques familiar to those skilled in the art.

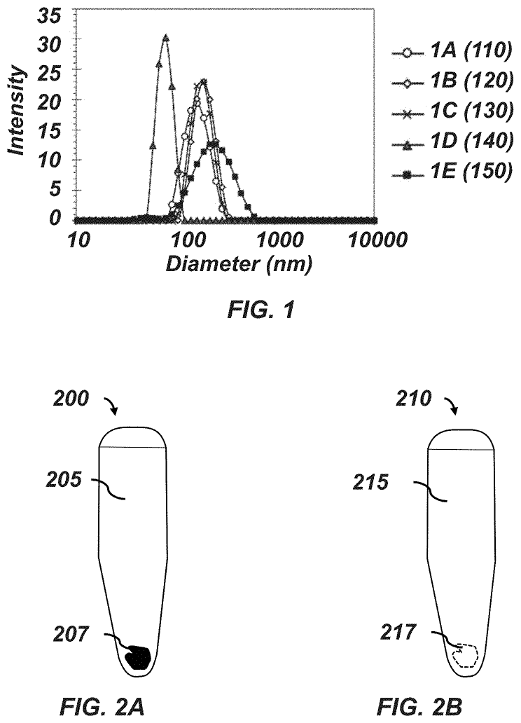

FIG. 1 is a graph showing Size distributions from dynamic light scattering of HPMCAS-stabilized nanoparticles containing Blue Dextran in the core.

FIGS. 2A and 2B are diagrams illustrating a microparticle pellet containing Blue Dextran under acidic conditions (water, FIG. 2A), and Blue Dextran release under neutral conditions (phosphate buffered saline at pH 7, FIG. 2B).

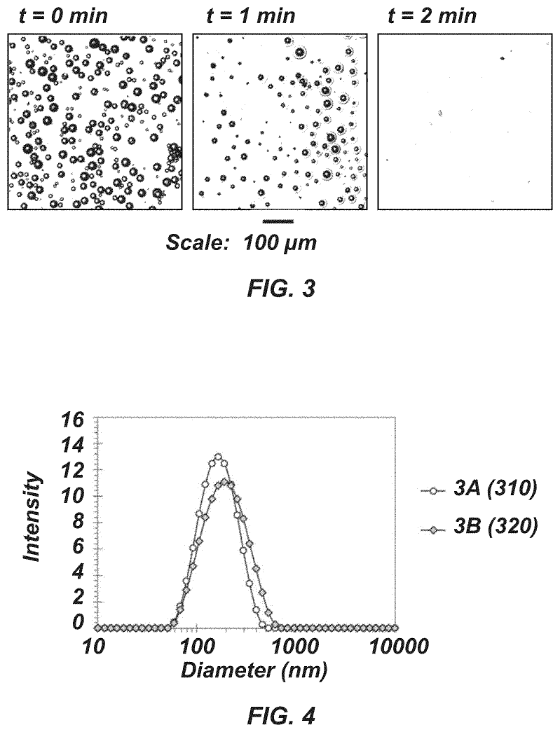

FIG. 3: Optical microscope time lapse showing Blue Dextran loaded microparticles falling apart over 2 minutes after being exposed to phosphate buffered saline.

FIG. 4: Size distributions from dynamic light scattering of HPMCAS-stabilized nanoparticles containing vancomycin in the core.

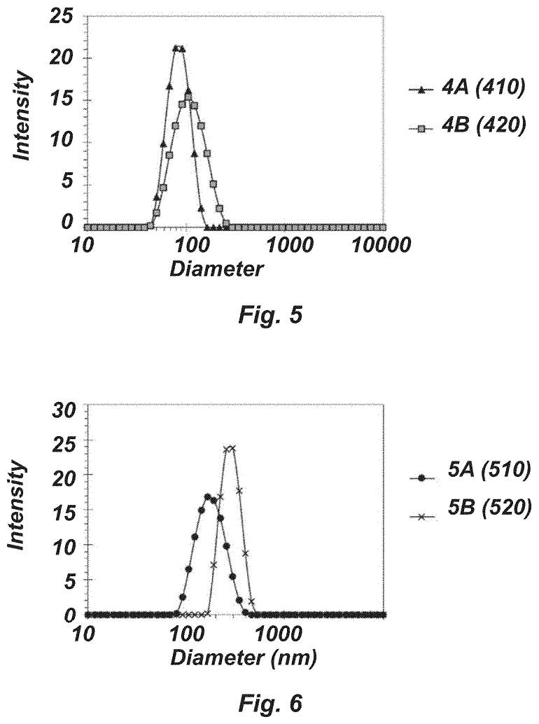

FIG. 5: Size distributions from dynamic light scattering of HPMCAS-stabilized nanoparticles containing Polymyxin B in the core.

FIG. 6: Size distributions from dynamic light scattering of HPMCAS-stabilized nanoparticles containing lysozyme in the core.

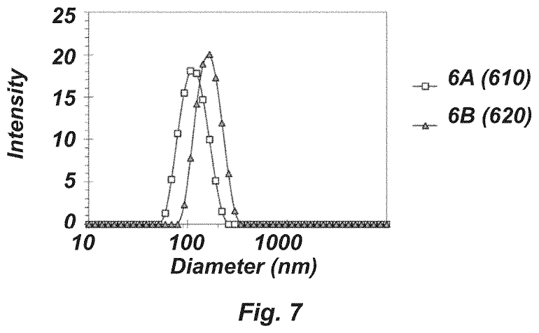

FIG. 7: Size distributions from dynamic light scattering of HPMCAS-stabilized nanoparticles containing horseradish peroxidase (HRP) in the core.

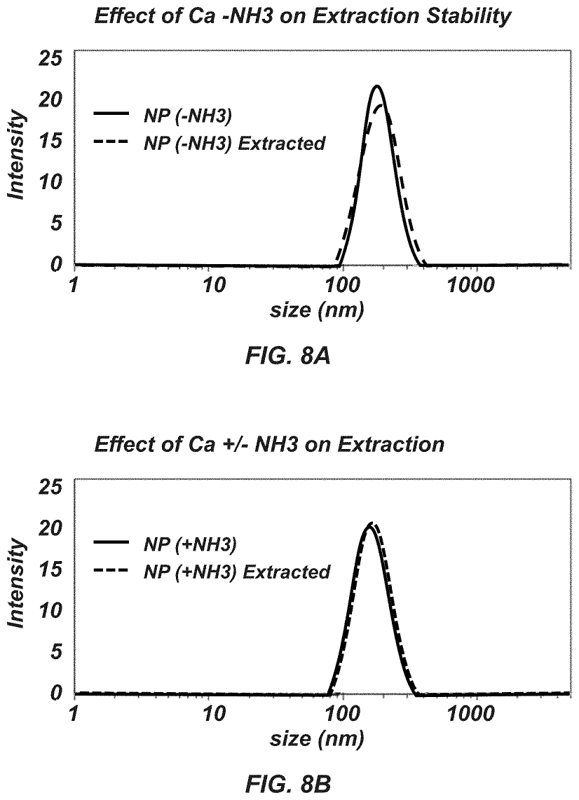

FIG. 8A: Size distributions from dynamic light scattering of Eudragit L100-55 stabilized nanoparticles containing maltodextrin in the core before and after extraction either without added ammonia.

FIG. 8B: Size distributions from dynamic light scattering of Eudragit L100-55 stabilized nanoparticles containing maltodextrin in the core before and after extraction with added ammonia.

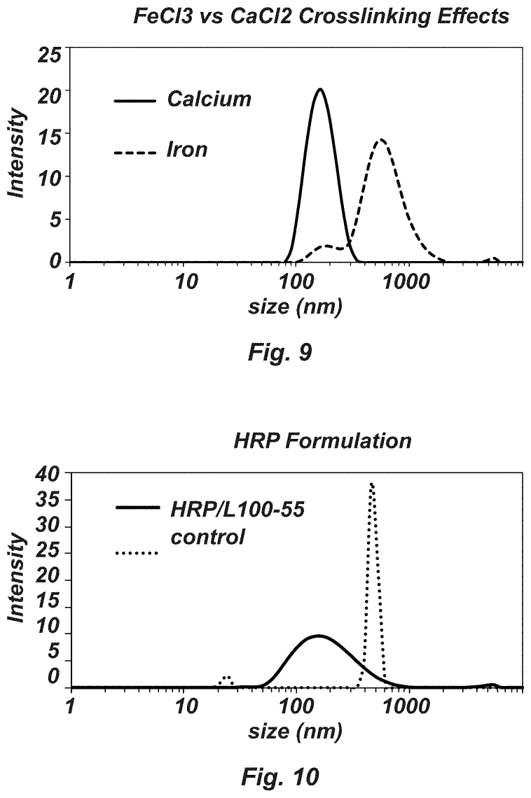

FIG. 9: Size distributions from dynamic light scattering of Eudragit L100-55 stabilized nanoparticles containing maltodextrin in the core with either CaCl₂ or FeCl₃ as a crosslinker.

FIG. 10: Size distributions from dynamic light scattering of Eudragit L100-55 stabilized nanoparticles containing HRP in the core as well as HRP aggregates without an Eudragit stabilizer.

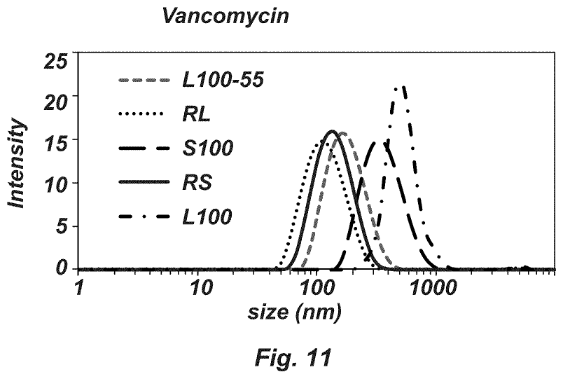

FIG. 11: Size distributions from dynamic light scattering of vancomycin-loaded nanoparticles with different Eudragit stabilizers.

### DETAILED DESCRIPTION

Embodiments are discussed in detail below. In describing embodiments, specific terminology is employed for the sake of clarity. However, the disclosure is not intended to be limited to the specific terminology so selected. A person skilled in the relevant art will recognize that other equivalent parts can be employed and other methods developed without parting from the spirit and scope of the disclosure. All references cited herein are incorporated by reference as if each had been individually incorporated.

Flash NanoPrecipitation (FNP): Flash NanoPrecipitation (FNP) is a process that combines rapid micromixing in a confined geometry of miscible solvent and antisolvent streams to effect high supersaturation of components. The resulting high supersaturation results in rapid precipitation and growth of the resulting nanoparticles. A stabilizing agent in the formulation accumulates on the surface of the nanoparticle and halts growth at a desired size. The process has been described in detail in Process and apparatuses for preparing nanoparticle compositions with amphiphilic copolymers and their use, BK Johnson, R K Prud'homme, U.S. Pat. No. 8,137,699, 2012. It has further been described in the review article by Saad and Prud'homme. (See D'addio, S. M.; Prud'homme, R. K., Controlling drug nanoparticle formation by rapid precipitation. Advanced drug delivery reviews 2011, 63 (6), 417-426; Johnson, B. K.; Prud′homme, R. K., Process and apparatuses for preparing nanoparticle compositions with amphiphilic copolymers and their use. Google Patents: 2012; Saad, W. S.; Prud′homme, R. K., Principles of nanoparticle formation by flash nanoprecipitation. Nano Today 2016, 11 (2), 212-227). These references are included in this application in their entirety. In Flash NanoPrecipitation the encapsulated agents are hydrophobic and the antisolvent is more polar than the antisolvent.

The Flash NanoPrecipitation process involves a confined mixing volume having one or more solvent streams entering the mixing volume, one or more antisolvent streams entering the mixing volume, and an exit stream (leaving the mixing volume) for the process. The velocity of the inlet streams into the confined mixing volume can be between about 0.01 m/s and 100 m/s, or about 0.1 m/s and 50 m/s, or about 0.1 m/s and 10 m/s. The velocities of the streams may be equal to one another, or they may have different velocities. In the case of unequal velocities, the velocity of the highest velocity stream is the specified velocity.

Inverse Flash NanoPrecipitation (iFNP): Inverse Flash NanoPrecipitation (iFNP) follows the same fundamental principles as Flash NanoPrecipitation. However, in iFNP the encapsulated agents are hydrophilic, and the antisolvent is more non-polar than the solvent. The inverse Flash NanoPrecipitation process is described in: World Patents WO/2015/200054 and WO 2017/112828 A1; Pagels, R. F.; Prud′homme, R. K., Polymeric nanoparticles and microparticles for the delivery of peptides, biologics, and soluble therapeutics. J Control Release 2015, vol. 219, 519-535; Pagels, R. F.; Prud′homme, R. K., Inverse Flash NanoPrecipitation for Biologics Encapsulation: Nanoparticle Formation and Ionic Stabilization in Organic Solvents. ACS Symposium Series 2017, Vol. 1271, Chapter 11, pp 249-274; Markwalter, C. E.; Prud'homme, R. K., Inverse Flash NanoPrecipitation for Biologics Encapsulation: Understanding Process Losses via an Extraction Protocol. ACS Symposium Series 2017, Vol. 1271, Chapter 12, pp 275-296. These references are included in this application in their entirety. Please note, that in some cases simply Flash Nanoprecipitation is used to refer to inverse Flash Nanoprecipitation. However, it should be clear from the encapsulated material, process solvent, and non-process solvent whether Flash NanoPrecipitation or inverse Flash NanoPrecipitation is being used.

The inverse Flash NanoPrecipitation process can be used to create “inverse” particles with hydrophilic cores and/or with encapsulated water-soluble agents, such as hydrophilic peptides. A stabilizing copolymer can be dissolved in a polar process solvent at a concentration of at least 0.1% by weight; the concentration of copolymer can be at least 0.2% by weight to form a first process solution. In an embodiment, the copolymer can be dissolved in the polar process solvent at a concentration in a range of from about 0.1 wt %, 0.2 wt %, 0.5 wt %, 1 wt %, 2 wt %, 5 wt %, 10 wt %, or 20 wt % to about 0.2 wt %, 0.5 wt %, 1 wt %, 2 wt %, 5 wt %, 10 wt %, 20 wt %, or 40 wt %. A person of skill in the art will appreciate that a factor such as the economics of a process can constrain a lower bound of concentration, and that factors such as the viscosity of the process solution or the solubility limit of the copolymer in the polar process solvent can constrain an upper bound of concentration. For example, if the viscosity of the first process solution is much greater than that of the nonprocess solvent, mixing of the first process solution with the nonprocess solvent may be inhibited. A person of skill in the art will appreciate that factors such as the molecular weight of the copolymer and the composition of the copolymer can affect the maximum concentration that can be attained in the polymer solution before the viscosity becomes too high. Examples of process solvents include, but are not limited to, water, alcohols, acetone, acetonitrile, glycol ethers, dimethyl sulfoxide (DMSO), dimethylformamide, N-methyl-2-pyrrolidone, and mixtures thereof. The process solvent can be heated or pressurized or both to facilitate dissolution of the polymer or hydrophilic active, depending on the dissolution characteristics of the copolymer in the solvent.

Upon micromixing the process solvent containing the copolymer with a less polar non-process solvent, such as chloroform, dichloromethane, or acetone, the dissimilar solubility characteristics of regions or portions of the copolymer are manifested, and the more polar portions of the copolymer can no longer exist in the soluble state, so that an “inverse” nanoparticle precipitates.

In an embodiment, additive water-soluble active agent, for example, a hydrophilic peptide, can be added to the copolymer in the process solvent. The concentration of the hydrophilic agent is typically within an order of magnitude of the concentration of the stabilizing polymer. If the concentration of the hydrophilic active is much lower than the concentration of the polymer than the final drug loading will be low. If the concentration of the hydrophilic active is much higher than the concentration of the polymer than there may not be enough stabilizing polymer to stabilize the nanoparticles. Upon creation of nanoparticles with the copolymer, the additive hydrophilic agent will be incorporated in the nanoparticle. Hydrophilic agents that are poorly soluble in the non-process solvent are coated, encapsulated, or confined as a particulate core and sterically stabilized by the protective colloid of the copolymer. The nanoparticles maintain a small and stable size in the nonprocess solvent.

In another embodiment, the hydrophilic agent and copolymer are dissolved in separate process solvent streams. The process solvent used to dissolve the copolymer and the process solvent used to dissolve the hydrophilic active material may be, but are not required to be, the same. For example, the target material (water soluble agent) can be dissolved in a first polar process solvent to form a water-soluble agent solution, and the copolymer can be dissolved in a second polar process solvent to form a copolymer solution. These streams, the water-soluble agent solution and the copolymer solution, are mixed, e.g., simultaneously mixed, with the nonprocess solvent to form a mixed solution. The first polar process solvent and the second polar process solvent can be miscible, or they can be completely miscible (i.e., so that another phase is not formed) at the volumetric ratios at which they are mixed. The first polar process solvent and the nonprocess solvent can be miscible, or they can be completely miscible (i.e., so that another phase is not formed) at the volumetric ratios at which they are mixed. The second polar process solvent and the nonprocess solvent can be miscible, or they can be completely miscible (i.e., so that another phase is not formed) at the volumetric ratios at which they are mixed. In another embodiment, the hydrophilic active material and copolymer are dissolved in a single process solvent stream. This stream is then rapidly mixed with a nonprocess solvent.

A person skilled in the art will recognize that all solvents are miscible to some degree in each other. Miscible solvents are used in the initial nanoparticle precipitation process. “Miscible” solvents as referred to herein are those that when mixed at the ratios used in the nanoparticle formation process or the microparticle process would produce solutions that have no more than 20% of the volume of the minor phase (e.g., a polar process solvent) not dissolved in the majority phase. Completely miscible solvents as referred to herein are those that when mixed at the ratios used in the nanoparticle formation process or the microparticle process would produce solutions with no phase separation “Immiscible” solvents as referred to herein are those that when mixed at the volume ratios used in the process produce less than 20% reduction in the volume of the minor phase due to solubilization into the majority phase.

The intense micromixing of the process solution and the non-process solvent can be affected in any number of geometries. The essential idea is that high velocity inlet streams cause turbulent flow and mixing that occurs in a central cavity. The time for process solvent/non-process solvent mixing is more rapid than the assembly time of the nanoparticles. While not meant to be limiting, two such geometries have been previously described and analyzed: The Confined Impinging Jet mixer (CIJ) (Johnson, B. K., Prud'homme, R. K. Chemical processing and micromixing in confined impinging jets. AIChE Journal 2003, 49, 2264-2282; Liu, Y., Fox, R. O. CFD predictions for chemical processing in a confined impinging-jets reactor. AIChE Journal 2006, 52, 731-744) and the multi-inlet vortex mixer (MIVM) (Liu, Y., Cheng, C., Liu, Y., Prud'homme, R. K., Fox, R. O. Mixing in a multi-inlet vortex mixer (MIVM) for flash nano-precipitation. Chemical Engineering Science 2008, 63, 2829-2842). These examples are meant to be illustrative rather than limiting or exhaustive.

The fast mixing and high energy dissipation involved in this process provide mixing timescales that are shorter than the timescale for nucleation and growth of particles, which leads to the formation of nanoparticles with active agent loading contents and size distributions not provided by other technologies. When forming the nanoparticles via Flash NanoPrecipitation, mixing occurs fast enough to allow high supersaturation levels, for example, as high as 10,000, of all components to be reached prior to the onset of aggregation. The supersaturation level is the ratio of the actual concentration of a material, for example, a copolymer, in a solvent to the saturation concentration of that material in that solvent. For example, the supersaturation levels can be at least about 1, 3, 10, 30, 100, 300, 1000, or 3000 and can be at most about 3, 10, 30, 100, 300, 1000, 3000, 10,000, 30,000, or 100,000. The timescales of aggregation of the hydrophilic active material and copolymer self-assembly are balanced. Therefore, the hydrophilic active material and polymers precipitate simultaneously, and overcome the limitations of low active agent incorporations and aggregation found with the widely used techniques based on slow solvent exchange (e.g., dialysis). The Flash NanoPrecipitation process is insensitive to the chemical specificity of the components, making it a universal nanoparticle formation technique.

The size of the resulting nanoparticles from this process can be controlled by controlling the mixing velocity used to create them, the total mass concentration of the copolymer and hydrophilic active molecules in the process solvent, the process and non-process solvents, the ratio of the copolymer and hydrophilic active molecule, and the supersaturation of the hydrophilic active molecule and non-soluble portion of the copolymer upon mixing with the non-process solvent.

Nanoparticles can be produced from copolymers that are dissolved in a process solvent with no hydrophilic active material added.

Using the disclosed methods, particles can be made that have sizes in the range of 15 nm to 10500 nm, sizes in the range of 20 nm to 6000 nm, sizes in the range of 20 nm to 1000 nm, sizes in the range of 35 nm to 400 nm, or sizes in the range of 40 nm to 300 nm. Sizes can be determined by dynamic light scattering. For example, particles can be made that have sizes of at least about 15 nm, 20 nm, 35 nm, 40 nm, 50 nm, 100 nm, 200 nm, 300 nm, 400 nm, 600 nm, 900 nm, 1000 nm, 2000 nm, 4000 nm, or 6000 nm, and have sizes of at most about 20 nm, 35 nm, 40 nm, 50 nm, 100 nm, 200 nm, 300 nm, 400 nm, 600 nm, 900 nm, 1000 nm, 2000 nm, 4000 nm, 6000 nm, or 10500 nm. Sizes reported and cited herein are the intensity average reported values as determined by the Malvern Nanosizer deconvolution program for particles smaller than 2000 nm, and determined by scanning electron microscopy, or optical microscopy and image analysis using Image J for sizes greater than 2000 nm. Other intensity weighted deconvolution methods can be used to determine sizes of the nanoparticles.

It was previously believed that inverse Flash NanoPrecipitation required the use of stabilizing block copolymers comprising distinct blocks or domains of hydrophobic separated from blocks or domains of hydrophilic blocks or domains. These were described and presented in the patent examples in WO 2017/112828 A1 and WO 2015/200054 as di-block copolymers and tri-block copolymers. With these block copolymers, the more hydrophilic/polar block(s) precipitated onto the nanoparticle surface, and the hydrophobic blocks remained soluble in the non-process solvent and form a steric shell. In WO 2015/200054, examples employed a diblock copolymer of poly(n-butyl acrylate)-b-poly(acrylic acid). Other polymers that have been used include poly(styrene)-b-poly(acrylic acid), poly(lactic acid)-b-poly(aspartic acid), and poly(ethylene glycol)-b-poly(lactic acid)-b-poly(aspartic acid). Claim 3 of WO 2015/200054 claims “the method of claim 1, wherein the copolymer is selected from the group consisting of a block copolymer, a diblock copolymer, a triblock copolymer, a multiblock copolymer, and a branched-comb copolymer.”

It was thought that if random polymers (e.g., random copolymers) were used, they could bridge between particles, so that stable nanoparticles would not be produced, and aggregation would result. That is, the steric stabilizing layer could need to have domains that anchor onto the nanoparticle surface, and the solvophilic domains could need to face out into the dispersing phase to provide a steric barrier to prevent particle-particle contacts. The particle bridging that occurs with inadequate distinction between hydrophilic and hydrophobic domains has been describe by Pham et al. and Horigome et al. (See, Pham. Q. T., et al. “Micellar solutions of associative triblock copolymers: Entropic attraction and gas-liquid transition.” Macromolecules 32.9 (1999): 2996-3005; and Horigome, Misao, and Yasufumi Otsubo. “Long-time relaxation of suspensions flocculated by associating polymers.” Langmuir 18.6 (2002): 19684973.) Furthermore, linear polymers and copolymers with random co-monomers can flocculate or aggregate particulate dispersions. (See, Halverson, Frederick. “Process for the flocculation of suspended solids.” U.S. Pat. No. 4,342,653. 3 Aug. 1982; Gregory, John, and Sandor Barany. “Adsorption and flocculation by polymers and polymer mixtures.” Advances in colloid and interface science 169.1 (2011): 1-12; and Mühle, K., and K. Domasch. “Stability of particle aggregates in flocculation with polymers: Stabilität von teilchenaggregaten bei der flockung mit polymeren.” Chemical Engineering and Processing: Process Intensification 29.1 (1991): 1-8.) Therefore, the results of the present application that randomly functionalized polymers were successfully used to formulate stable nanoparticles that did not aggregate was unexpected.

Descriptions of Actives (Encapsulated Material)

An active is the component or material which confers the desired performance or result. This may be a pharmaceutical active (e.g., a drug, a therapeutic, or a diagnostic (e.g., tracing) material), a fragrance, a cosmetic, a pesticide, an herbicide, an ink or a dye, a molecule or composition that enables covert security labeling, or a molecule or composition that registers a change in color when undergoing some process event. In this document the terms hydrophilic active, hydrophilic agent, and target are used interchangeably.

Encapsulated actives (target molecules) must be sufficiently polar that they rapidly precipitate in the less polar non-process solvent. Molecules that do not meet these criteria may be chemically modified to increase their water solubility and propensity to precipitate in the organic non-process solvent. Examples of biologic material that may be encapsulated include, but are not limited to, peptides, proteins, DNA, RNA, saccharides, and derivatives, conjugates, and/or analogs thereof. Small molecule water soluble therapeutics and imaging agents may also be encapsulated. Soluble stabilizing agents may be encapsulated in particles to provide stability to the particle for its use or for subsequent processing steps. Any of these materials may also be co-precipitated within a single particle. Hydrophilic material may be encapsulated for the sole purpose of adding stability to the particles during post processing. For example, material with molecular weights between 100 and 10,000,000 Daltons (Da) may be encapsulated. Material with molecular weights between 250 and 10,000,000 Da may be encapsulated. Material with molecular weights between 100 and 1,000,000 Da may be encapsulated. Material with molecular weights between 250 and 1,000,000 Da may be encapsulated. Material with molecular weights between 100 and 200,000 Da may be encapsulated.

Certain encapsulated materials may be multifunctional. For example, tobramycin is cationic and can itself be crosslinked with a copolymer. Other cationic active materials, with multiple cationic residues will similarly crosslink the anionic polymer blocks.

The encapsulated material may be incorporated into the particle at a range of loadings. For example, the mass of the encapsulated material may be greater than or equal to the mass of the copolymer. For example, the concentration of the encapsulated material in the first process solution may be from about 0.1 wt %, 0.2 wt %, 0.5 wt %, 1 wt %, 2 wt %, 5 wt %, 10 wt %, or 20 wt % to about 0.2 wt %, 0.5 wt %, 1 wt %, 2 wt %, 5 wt %, 10 wt %, 20 wt %, or 40 wt %.

Description of Stabilizing Random Copolymers

In this disclosure, random copolymers are used as stabilizers in the inverse Flash Nanoprecipitation process. The random copolymers are composed of at least 2 different monomers, chemical moieties, or components. To be used as stabilizers in the iFNP process at least one or more of the polymer components should be non-polar. The non-polar moiety may be a non-polar monomer, or it may be a monomer or polymer component that has been made non-polar by chemical reaction or modification of the moiety either before or after polymer synthesis. For example, an anhydro-glucose unit on a polysaccharide chain may have one of more of the hydroxyl groups on the ring reacted with a non-polar chemical species. For example, the random copolymer can be greater than 2 wt %, 5 wt %, 10 wt %, 20 wt %, 50 wt %, 60 wt %, 70 wt %, 80 wt %, 90 wt % or 95 wt % non-polar. These non-polar groups should be soluble in the non-process solvent. To be used as stabilizers in the iFNP process at least one or more of the polymer components should be polar. For example, the random copolymer can be greater than 2 wt %, 5 wt %, 10 wt %, 20 wt %, 50 wt %, 60 wt %, 70 wt %, 80 wt %, 90 wt % or 95 wt % polar. These polar groups should be insoluble in the non-process solvent. During the iFNP process, the polar groups should precipitate in the non-process solvent and the non-polar group should remain soluble.

In one embodiment, the random copolymer is cellulosic. Cellulose-based random copolymers can also include: cellulose acetate phthalate (cellacefate), cellulose acetate succinate, hydroxypropyl methyl cellulose phthalate, hydroxypropyl methyl cellulose acetate succinate (HPMCAS, hypromellose acetate succinate), and cellulose acetate trimellitate. HPMCAS is a cellulosic polymer of a cellulose ester bearing acetyl and succinyl groups. It is synthesized by the esterification of HPMC with acetic anhydride and succinic anhydride, which offers flexibility in acetate and succinate substitution levels, and which allows for optimization of both solubility enhancement and material processing. HPMCAS has been used to maintain stable solid dispersions and inhibit drug crystallization through spray-dried dispersion or hot melt extrusion. The world patent application WO/2019/055539 describes the use of HPMCAS as a stabilizer to encapsulate hydrophobic actives in an aqueous external phase. However, no studies describe nanoparticle formulation with HPMCAS as the surface stabilizer for an inverse Flash NanoPrecipitation process in a non-aqueous external phase. HPMCAS 126 can have a hydroxypropyl substitution of from 6 to 10%, a methoxy substitution of from 22 to 26%, an acetate (acetyl) substitution of from 10 to 14%, and a succinate (succinyl) substitution of from 4 to 8%. HPMCAS 716 can have a hydroxypropyl substitution of from 5 to 9%, a methoxy substitution of from 20-24%, an acetate (acetyl) substitution of from 5 to 9%, and a succinate (succinyl) substitution of from 14 to 18%. HPMCAS 912 can have a hydroxypropyl substitution of from 5 to 9%, a methoxy substitution of from 21 to 25%, an acetate (acetyl) substitution of from 7 to 11%, and a succinate (succinyl) substitution of from 10 to 14%. The hydroxypropylmethylcellulose—acetate succinate polymers can have hydroxypropyl substitution levels of 5-10% wt, methoxy substitution levels of 20-26% wt, acetyl substitutions of 5-14% wt (such as 10-14% wt substitution), and succinyl substitutions of 4-18% wt (such as 4-8% wt). Polymers of the present disclosure can have weight average molecular weights between about 10,000-2,000,000 g/mol, or about 10,000-500,000 g/mol, or about 20,000-400,000 g/mol, or about 50,000-250,000 g/mol. HPMC E3 (METHOCEL E3 Dow Chemical Company) can have a hydroxypropyl substitution of from 7-12% and a methoxyl substitution of from 28-30%.

For example, the cellulosic polymer can be hydroxypropyl cellulose, methyl cellulose, ethyl methyl cellulose, hydroxypropylmethylcellulose, carboxymethyl cellulose, or a combination of these. The cellulosic polymer can include hydroxypropyl, hydroxyethyl, hydroxymethyl, succinate, and/or acetate substitution(s).

For example, the cellulosic polymer can be hydroxypropylmethyl cellulose including a hydroxypropyl substitution level of from 5 to 10% wt. For example, the cellulosic polymer can be hydroxypropylmethyl cellulose including a methoxyl substitution level of from 20 to 26% wt. For example, the cellulosic polymer can be hydroxypropylmethyl cellulose including an acetyl substitution level of from 5 to 14% wt or from 10 to 14% wt. For example, the cellulosic polymer can be hydroxypropylmethyl cellulose including a succinyl substitution level of from 4 to 18% wt or from 4 to 8% wt.

In another embodiment of the stabilizing random copolymer is not cellulosic.

For example, poly(meth)acrylate-based random copolymers may be used. Poly(meth)acrylate-based random copolymers are frequent oral excipients that impart specific functionalities when used as coatings or matrices. Eudragit polymers produced by Evonik Industries are one commercialized version of these. The reported characteristics of different copolymers in the Evonik oral excipients line are summarized in Table 1. The first row provides the brand name, which will be used throughout the text. The average MW column reports the weight averaged molar mass based on a poly(methylmethacrylate) standard. The random copolymers used in enteric applications consist of a mixture of monomers shown in the final column, generally containing methyl or ethyl ester sidechains as well as monomers of methacrylic acid. The relative compositions are varied to tune the pH responsiveness. The RS and RL forms contain a quaternary amine side chain in lieu of the anionic functionality (ammonio methacrylate copolymer) while Eudragit E contains a tertiary amine (dimethylaminoethyl methacrylate-butyl methacrylate-methyl methacrylate).

Some Eudragits have been used in an aqueous environment to encapsulate peptides. For example, Eudragit L100 and RSPO have been used to form nanoparticles containing salmon calcitonin. This was achieved by adding organic solvent solutions containing the Eudragit to an aqueous solution containing poly(vinyl alcohol) and salmon calcitonin. (See Cetin et al. Salmon calcitonin-loaded Eudragit and Eudragit-PLGA nanoparticles: in vitro and in vivo evaluation. J Microencapsulation. 2011.) These examples are quite distinct from the presently disclosed methods, which are carried out using a non-polar solvent as the external phase of the final nanoparticles. Using the external aqueous phase, as described by Cetin et al., results in the loss of the aqueous soluble active into the aqueous phase, and the poor capture of the active into the hydrophobic phase. In sharp contrast, the external phase in the encapsulation process disclosed herein is a non-solvent for the aqueous soluble active. Therefore, essentially none of the aqueous soluble active is lost into the non-polar external phase. The result is very high encapsulation efficiencies.

TABLE 1
Examples of random copolymers used in oral drug delivery.

| | | | Avg | Notes & Monomer | |
|---|---|---|---|---|---|
| Name | Use | pH/Target | MW | Constituents | |
| L100-55 | Enteric | ≥5.5 Duodenum | 320,000 | Anionic. | 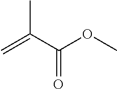 |
| L100 | Enteric | ≥6.0 Jejunum | 125,000 | Anionic. | 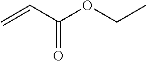 |
| S100 | Enteric | ≥7.0 Ileum/colon | 125,000 | Anionic. | 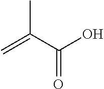 |
| FS 30 D (aqueous dispersion) | Enteric | ≥7.0 Ileum/colon | 280,000 | Anionic | 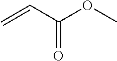 |

| | | | | |
|---|---|---|---|---|
| NE 40D | Sustained | swell in | 750,000 | Not charged; can be |
| (aq. disp.) | release | physiological | | combined with both ionic |
| | | media | | and non-ionic active |
| | | | | ingredients (neutral ester) |
| NM 30D | Sustained | swell in | 600,000 | Not charged; can be |
| (aq. disp.) | release | physiological | | combined with both ionic |
| | | media | | and non-ionic active |
| | | | | ingredients (neutral for |
| | | | | matrix) |
| RL PO | Sustained | swell in | 32,000 | Cationic; when w/anionic |
| (powder) | release | physiological | | APIs, ionic interactions may |
| | | media | | increase retardation |
| RS PO (powder) | Sustained release | swell in physiological media | 32,000 | 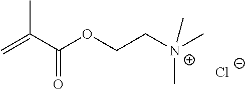 |
| | | | | 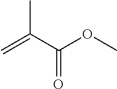 |
| | | | | 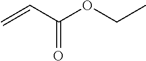 |
| E PO | Taste/odor | Soluble in | 47,000 | Cationic |
| (powder) | masking | gastric fluid up to pH 5.0; swells above pH 5.0 | | 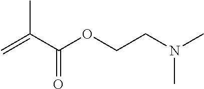 |
| E 100 (granules) | Taste/odor masking | Soluble in gastric fluid up to pH 5.0; swells above pH 5.0 | 47,000 | 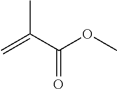 |
| | | | | 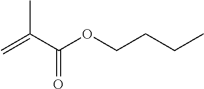 |

Other commercialized forms of poly(meth)acrylate polymers for oral delivery exist. For example, BASF produces assorted classes of Kollicoat polymers which match the regulatory monograph for the equivalent Eudragit polymers for both enteric and taste masking applications. While examples provided herein were carried out with Eudragit polymers, the performance relies on chemical characteristics and not the brand and should not be construed as being limited to the specific polymer source.

Other random copolymers with suitable stabilizing characteristics used for oral delivery of therapeutics include but are not limited to: vinyl acetate-vinylpyrrolidone copolymer (for example, Kollidon VA 64) and polyvinyl acetate phthalate (Phthalavin).

Stabilizing random copolymers can be copolymers of hydrophilic and hydrophobic amino acids. Stabilizing random copolymers can be copolymers of amino acids with other monomers. For example, poly(aspartic acid-co-lactic acid) can be used as a stabilizing random copolymer.

Examples of suitable nonpolar components of a random copolymer include but are not limited to the following: acrylates including methyl acrylate, ethyl acrylate, propyl acrylate, n-butyl acrylate (BA), isobutyl acrylate, 2-ethyl acrylate, and t-butyl acrylate; methacrylates including ethyl methacrylate, n-butyl methacrylate, and isobutyl methacrylate; acrylonitriles; methacrylonitrile; vinyls including vinyl acetate, vinylversatate, vinylpropionate, vinylformamide, vinylacetamide, vinylpyridines, vinyl phenols and vinyllimidazole; aminoalkyls including aminoalkylacrylates, aminoalkylsmethacrylates, and aminoalkyl(meth)acrylamides; styrenes; cellulose acetate phthalate, cellulose acetate succinate, hydroxypropylmethylcellulose phthalate, poly(D,L-lactide), poly (D,L-lactide-co-glycolide), poly(glycolide), poly(hydroxybutyrate), poly(alkylcarbonate) and poly(orthoesters), polyesters, poly(hydroxyvaleric acid), polydioxanone, poly(ethylene terephthalate), poly(malic acid), poly(tartronic acid), polyanhydrides, polyphosphazenes, poly(amino acids), lactic acid, caprolactone, glycolic acid, and their copolymers (see generally, Illum, L., Davids, S. S. (eds.) Polymers in Controlled Drug Delivery Wright, Bristol, 1987; Arshady, J. Controlled Release 17:1-22, 1991; Pitt, Int. J. Phar. 59:173-196, 1990; Holland et al., J. Controlled Release 4:155-0180, 1986); hydrophobic peptide-based polymers and copolymers based on poly(L-amino acids) (Lavasanifar, A., it al., Advanced Drug Delivery Reviews (2002) 54:169-190), poly(ethylenevinyl acetate) (“EVA”) copolymers, silicone rubber, polyethylene, polypropylene, polydienes (polybutadiene, polyisoprene and hydrogenated forms of these polymers), maleic anhydride copolymers of vinyl methylether and other vinyl ethers, polyamides (nylon 6,6), polyurethane, poly(ester urethanes), poly(ether urethanes), poly(esterurea). For example, polymeric blocks can include poly(ethylenevinyl acetate), poly(D,L-lactic acid) oligomers and polymers, poly(L-lactic acid) oligomers and polymers, poly(glycolic acid), copolymers of lactic acid and glycolic acid, poly(caprolactone), poly(valerolactone), polyanhydrides, copolymers of poly(caprolactone) or poly(lactic acid), or poly(propylene sulfide). For non-biologically related applications polymeric blocks can include, for example, polystyrene, polyacrylates, and butadienes.

Natural products with sufficient hydrophobicity to act as the non-polar portion of the random copolymer include: hydrophobic vitamins (for example vitamin E, vitamin K, and vitamin A), carotenoids, and retinols (for example, beta carotene, astaxanthin, trans and cis retinal, retinoic acid, folic acid, dihydrofolate, retinylacetate, retinyl palmintate), cholecalciferol, calcitriol, hydroxycholecalciferol, ergocalciferol, alpha-tocopherol, alpha-tocopherol acetate, alphatocopherol nicotinate, estradiol, lipids, alcohols with carbon numbers from 12 to 40, cholesterols, unsaturated and/or hydrogenated fatty acids, salts, esters or amides thereof, fatty acids mono-, di- or triglycerides, waxes, ceramides, cholesterol derivatives or mixtures thereof. For example, a natural product is vitamin E which can be readily obtained as a vitamin E succinate, which facilitates functionalization to amines and hydroxyls on the active species.

For example, a hydrophilic polymer can be modified with hydrophobic small molecules to for a stabilizing random copolymer. For example, some of the acid groups of poly(acrylic acid), poly(aspartic acid), or poly(glutamic acid) could be esterified with hydrophobic alcohols such as an aliphatic alcohol to form a stabilizing random copolymer.

A suitable polar component of the stabilizing copolymer is insoluble in the nonprocess solvent. The following maybe used or used in combinations, and includes but is not limited to the following: carboxylic acids including acrylic acid, methacrylic acid, itaconic acid, and maleic acid; polyoxyethylenes or polyethylene oxide; polyacrylamides and copolymers thereof with dimethyl-aminoethyl-methacrylate, diallyl-dimethyl-ammonium chloride, vinylbenzyl trimethylammonium chloride, acrylic acid, methacrylic acid, 2-acryamideo-2-methylpropane sulfonic acid and styrene sulfonate, polyvinyl pyrrolidone, starches and starch derivatives, dextran and dextran derivatives; polypeptides, such as polylysines, polyarginines, polyaspartic acids, polyglutamic acids; poly hyaluronic acids, alginic acids, polylactides, polyethyleneimines, polyionenes, polyacrylic acids, and polyiminocarboxylates, gelatin, and unsaturated ethylenic mono or dicarboxylic acids. Others include polyoxyethylenes, poly(ethylene glycol), poly(propylene oxide), polysaccharides, poly(vinyl alcohol), polypeptides, polyvinyl pyrrolidone, starches and starch derivatives, dextran and dextran derivatives, gelatin, DMAEMA (dimethyl aminoethyl methacrylate), polyvinyl pyridine (PVP), and/or dimethyl aminoethyl acrylamide (DMAMAM), poly(N-(2-Hydroxypropyl) methacrylamide), or combinations.

To prepare anionic copolymers, acrylic acid, methacrylic acid, poly(glutamic acid) and/or poly aspartic acid polymers can be used. To produce cationic copolymers, DMAEMA (dimethyl aminoethyl methacrylate), polyvinyl pyridine (PVP), and/or dimethyl aminoethyl acrylamide (DMAMAM) can be used. A listing of suitable polar, water soluble, polymers can be found in Handbook of Water-Soluble Gums and Resins, R. Davidson, McGraw-Hill (1980).

The lists above of nonpolar and polar polymer components should not be considered exclusive of one another. Copolymers of two polymers given in a single list may have sufficient differences in solubilities in a given nonprocess solvent to be used in this process.

Stabilizing random copolymers can have molecular weights ranging from about 0.1 kDa, 1 kDa, 10 kDa, 100 kDa, or 1000 kDa to about 5 kDa, 50 kDa, 100 kDa, 1000 kDa or greater.

Description of Process and Non-Process Solvents

Formation of nanoparticles requires one or more process solvents and one or more non-process solvent streams. The process and non-process solvents may be a pure (that is, a single) liquid compound or a mixture of two or more pure liquid compounds. Other non-liquid compounds that aid in the solvent quality of the streams may be added and are also considered part of the solvent. For example, a surfactant, a salt, or a cosolvent may be added to a solvent and considered part of the solvent. These excipient compounds may or may not be in the final nanoparticle or microparticle construct, depending on the requirements of the final product.

The polar process solvent containing the copolymer is chosen such that the copolymer is molecularly dissolved. This requires that the process solvent solubilize all parts of the copolymer. The process solvent containing the material to be encapsulated, if present, is also chosen such that material is molecularly dissolved. These process solvents may be, but are not required to be, the same. In some cases, both the copolymer and material to be encapsulated may be dissolved in a single solution of the process solvent. In order to dissolve the water-soluble material to be encapsulated, the process solvent is more polar than the non-process solvent. Examples of process solvents include, but are not limited to, water, alcohols, methanol, ethanol, glycol ethers, dimethyl formamide (DMF), dimethyl sulfoxide (DMSO), acetonitrile, acetone, N-methyl pyrrolidone (NMP), and mixtures thereof. Acids, bases, and salts are a few examples of additives that may be used to aid in the solubilization of the copolymer and encapsulated material in the process solvent.

The solutions of process solvent containing copolymer and material to be encapsulated are mixed with a nonprocess solvent. The non-process solvent must be capable of changing the local molecular environment of the copolymer and causing local precipitation of the hydrophilic components of the copolymer. The nonprocess solvent is chosen such that the more polar sections of the copolymer rapidly precipitate while the more non-polar components of the copolymer remain solubilized. Thus, the copolymer will self-assemble into the desired nanoparticle form in the nonprocess solvent. The nonprocess solvent is chosen such that the hydrophilic active material to be encapsulated rapidly precipitates in the final mixture. In most cases it is preferable for the process and non-process solvents to be fully miscible at the final composition. In some cases, no more than 20 volume percent of the process solvent may phase separate in the final composition. In general, this is only acceptable if the phase separated solvent goes to the core of the particles and there is no macroscopic separation. For example, some water in the process solvent may phase separate from the non-process solvent and segregate into the hydrophilic nanoparticle core. Non-process solvents include, but are not limited to, chloroform, dichloromethane, alkanes such as hexane, ethers such as diethyl ether, ethyl acetate, tetrahydrofuran (THF), toluene, acetone, and mixtures thereof. Acids, bases, and salts are a few examples of additives that may be used to aid in the precipitation of the encapsulated material and sections of the copolymer. Solvent choices are made based on the solubilities of the copolymer and encapsulated materials. It is important to note that process solvents of one system may work well as the nonprocess solvent in another system, thus the examples given above for process and nonprocess solvents should not be considered distinct.

Crosslinking of Nanoparticles

An embodiment of the disclosed method includes stabilizing the nanoparticle core through crosslinking of the copolymer. For example, the nanoparticle can be crosslinked during assembly of the nanoparticle. For example, the nanoparticle can be crosslinked after assembly of the nanoparticle. The crosslinking can be covalent crosslinking. For example, the crosslinking can be disulfide crosslinking. The crosslinking can involve as cleavable ester linkage of the types described in US patent application Ser. No. 13/969,449, Particulate Constructs for Release of Active Agents, Lawrence Mayer, et al. The crosslinking can be non-covalent. For example, the crosslinking can be ionic, chelation, acid-base, or hydrogen bonding crosslinking.

A crosslinking agent can be added to crosslink the copolymer. For example, the crosslinking agent can be added to crosslink groups of the copolymer having anionic functionality or character. For example, the crosslinking agent can be an alkaline earth halide, a magnesium halide, magnesium chloride, a calcium halide, calcium chloride, a transition metal halide, an iron halide, iron(III) chloride, spermine, or combinations. For example, the crosslinking agent can be a metal acetate, an alkaline earth acetate, a transition metal acetate, calcium acetate, or combinations. For example, the crosslinking agent can be chromium(III) acetate, or another chromium (III) salt. For example, the crosslinking agent can be a metal nitrate, an alkaline earth nitrate, a transition metal nitrate, calcium nitrate, zinc nitrate, iron nitrate, or combinations. Other bio-compatible multi-cationic water-soluble agents may be used as crosslinking agents, for example, to crosslink anionic sections of the copolymer. For example, the water-soluble agent can include tobramycin and the tobramycin can crosslink the copolymer. One example is tetraethylene pentamine Ammonia or another chemical with basic character can be added to promote ionic interactions between the cationic crosslinker and the hydrophilic groups of the copolymer, if they have anionic functionality.

Further Processing of Inverted Nanoparticles

After nanoparticle formation, additional processing steps can be carried out to generate a desirable formulation. Residual DMSO can be removed using an extraction process if the nanoparticles are dispersed in a water immiscible solvent. The composition of the aqueous solution can be modified to promote stability of the polymer stabilizer. For example, the aqueous stream may contain 150 mM sodium chloride to tune the osmolarity. A sugar, a PEG, or other osmolyte may be used to achieve a similar effect. The pH can be adjusted to limit stabilizer solubility. Organic acids like acetic acid or citric acid can be used, as can mineral acids such as hydrochloric acid. Similarly, bases such as ammonium hydroxide and sodium hydroxide can be added. Buffer systems such as borate or carboxylate or others may be used. The extraction can be carried out for 30 minutes or longer using standard protocols acceptable to the processing scale. The aqueous phase along with the interface is sent to waste and the organic phase is retained for further processing. This is described in Markwalter, C. E.; Prud'homme, R. K., Inverse Flash NanoPrecipitation for Biologics Encapsulation: Understanding Process Losses via an Extraction Protocol. ACS Symposium Series 2017, Vol. 1271, Chapter 12, pp 275-296, which is included in its entirety.

Several different processing routes may be envisioned depending upon the formulation requirements. In Route 1, the immiscible solvent phase can be emulsified in an external aqueous phase containing a surfactant. For example, the nanoparticle phase can be emulsified in water containing poly(vinyl alcohol). Hydrophobic additives including small molecules or polymers may be added to the nanoparticle oil phase. After emulsification the solvent can be removed to form microparticles. This process is described in World Patent WO 2017/112828 A1; Pagels, R. F.; Prud'homme, R. K., Polymeric nanoparticles and microparticles for the delivery of peptides, biologics, and soluble therapeutics. J Control Release 2015, vol. 219, 519-535; Pagels, R. F.; Prud'homme, R. K., Inverse Flash NanoPrecipitation for Biologics Encapsulation: Nanoparticle Formation and Ionic Stabilization in Organic Solvents. ACS Symposium Series 2017, Vol. 1271, Chapter 11, pp 249-274. These references are included in this application in their entirety.

In Route 2, the nanoparticles may be recovered directly from drying of the organic solvent to form a powder and processed by traditional routes such as tableting with additional excipients. In Route 3, the nanoparticles are spray dried (from the original organic solvent or after solvent exchange) with additional excipients to form microparticles.

Frequently, it is desirable to adjust the organic stream before proceeding through Route 1 processing. Solvent can be evaporated to generate a more concentrated nanoparticle dispersion. Additional polymer can be added as a binder, glue, or property modifier. For example, additional enteric polymers can be added before emulsification. The polymer may be the same as the nanoparticle stabilizer or it may be different. The emulsification forms a nanoparticle-in-oil-in-water system. The surfactant stabilizer can be a poly(vinyl alcohol), a pluronic, a Tween, or another stabilizer. Additives may be included in the aqueous phase such as salts, sugars, pH modifiers, osmolytes, or organic solvents to achieve specific functions. The emulsification can be carried out using techniques familiar to those skilled in the art. Removal of the residual organic phase can be achieved by evaporation under suitable conditions. The particles may be washed and lyophilized.

Route 3 is an alternative method to form microparticles that employs spray drying. Frequently, additional excipients such as additional polymer may be necessary to include. In this case, if the original organic solvent limits solubility, it may be necessary to carried out a solvent exchange to a suitable solvent such as tetrahydrofuran (THF). Crosslinking of the nanoparticle stabilizer ensures colloidal stability in this step and at the same time allows free polymer to be added prior to the spray drying step. Spray drying may be carried out using techniques that familiar to those skilled in the art.

### EXAMPLES

Three HPMCAS polymers with different substitution ratios of succinyl and acetyl groups were used in Flash NanoPrecipitation of a new nanoparticle formulation. AFFINISOL™ Hypromellose acetate succinate (HPMCAS) 126, 716, 912 polymers were donated from Dow Chemical Company (Midland, Mich.). HPMCAS 126 can have a hydroxypropyl substitution of from 6 to 10%, a methoxyl substitution of from 22 to 26%, an acetate (acetyl) substitution of from 10 to 14%, and a succinate (succinyl) substitution of from 4 to 8%. HPMCAS 716 can have a hydroxypropyl substitution of from 5 to 9%, a methoxyl substitution of from 20-24%, an acetate (acetyl) substitution of from 5 to 9%, and a succinate (succinyl) substitution of from 14 to 18%. HPMCAS 912 can have a hydroxypropyl substitution of from 5 to 9%, a methoxyl substitution of from 21 to 25%, an acetate (acetyl) substitution of from 7 to 11%, and a succinate (succinyl) substitution of from 10 to 14%. HPMCAS 126 has the highest acetyl substitution and is the most hydrophobic, and HPMCAS 716 is the most hydrophilic, with the highest succinyl substitution. In the following, several examples of successful encapsulation of a variety of active pharmaceutical ingredients with nanoparticles stabilized by HPMCAS are provided.

Additionally, commercially-available methacrylate-based random copolymers were used to formulate biologics in an inverse Flash NanoPrecipitation process. Eudragit polymers were supplied by Evonik. The six polymers tested represent a range of physical properties based upon the monomer make-up of each type as summarized in Table 1.

### Example 1: Blue-Dextran Loaded HPMCAS Nanoparticles

Blue dextran (BD) with a nominal molecular weight of 2,000,000 Da was encapsulated in inverted nanoparticles stabilized by HPMCAS, and were prepared by inverse Flash NanoPrecipitation (iFNP). The process solvent stream was comprised of dimethyl sulfoxide (DMSO) with BD, HPMCAS (126, 912, or 716), NaOH, and a small volume percentage of water. The compositions of the process solvent streams are given in Table 2. The non-process solvent was dichloromethane (DCM).

The NaOH is added to the process solvent to form the sodium salt of the succinate groups on the HPMCAS. The sodium salt is more hydrophilic than the free acid, and helps these groups precipitate when exposed to the non-process solvent. To calculate the equivalents of NaOH, the HPMCAS 716 was assumed to be 16 wt % succinate, the HPMCAS 912 was assumed to be 12 wt % succinate, and the HPMCAS 126 was assumed to be 6 wt % succinate.

The process solvent (500 μL) and non-process solvent (500 μL) were rapidly mixed in a confined impingement jets (CIJ) mixer, and the effluent was collected in 4 mL of DCM. Samples 1A, 1B, 1C, 1D, and 1E formed translucent solutions with no visible aggregates. The control with no HPMCAS stabilizer, Sample 1F, resulted in the immediate formation of visible aggregates of BD.

TABLE 2
The process solvent stream compositions for Example 1.
In all cases the solvent was DMSO. The NaOH equivalents
are with respect to the acid groups on the HPMCAS.

| | | HPMCAS | BD | | NaOH | |
|---|---|---|---|---|---|---|
| | | Concen- | Concen- | | Concen- | NaOH |
| | HPMCAS | tration, | tration, | Water | tration, | equiv- |
| Sample | Used | mg/mL | mg/mL | content | μg/mL | alents |
| 1A | 126 | 5 | 5 | 5 v % | 60 | 0.5 |
| 1B | 912 | 5 | 5 | 5 v % | 120 | 0.5 |
| 1C | 716 | 5 | 5 | 5 v % | 160 | 0.5 |
| 1D | 126 | 5 | 5 | 5 v % | 90 | 0.75 |
| 1E | 126 | 2.5 | 7.5 | 10 v % | 30 | 0.5 |
| 1F | n/a | 5 | 0 | 5 v % | 0 | n/a |

Nanoparticle size distributions were measured by dynamic light scattering (DLS) in DCM. All three of the HPMCAS polymers resulted in nanoparticles ranging from 80 nm to 250 nm, and the PK1 diameter and the polydispersity index (PDI) from the Malvern Zetasizer DLS software are given in Table 3 and the size distributions are given in FIG. 1, showing distributions for 1A (110), 1B (120), 1C (130), 1D (140), and 1E (150).

TABLE 3
Particle diameters and PDIs for all samples in Example 1.

| | Diameter, | |
|---|---|---|
| Sample | nm | PDI |
| 1A | 170 | 0.03 |
| 1B | 195 | 0.16 |
| 1C | 185 | 0.02 |
| 1D | 80 | 0.18 |
| 1E | 250 | 0.17 |
| 1F | n/a | n/a |

The HPMCAS polymer did not greatly impact the nanoparticle size in this example (see Samples 1A, 1B, and 1C for HPMCAS 126, 912, and 716, respectively, in Table 3). Increasing the amount of NaOH from 0.5 equivalents in Sample 1A to 0.75 equivalents in Sample 1E resulted in a decrease in nanoparticle size. Finally, increasing BD loading from ˜50 wt % in Sample 1A to ˜75% in sample 1D resulted in an increase in nanoparticle size.

### Example 2: Incorporation of Blue-Dextran Loaded HPMCAS Nanoparticles into HPMCAS Microparticles

The inverted nanoparticles of Sample 1A given in Example 1 were embedded into HPMCAS microparticles through an emulsion process. The DMSO in the inverted nanoparticle dispersion was extracted with 2 mL of 150 mM NaCl in water. The brine solution was added to the top of the DCM phase and the vial was inverted 5 times and then gently shaken for 30 min., after which the high density DCM oil phase containing the inverted nanoparticles was removed. The inverted nanoparticles were concentrated to −10 mg/mL by rotary evaporating. A 10 mg/mL solution of HPMCAS 126 in DCM with 10 v % methanol was produced. Equal volumes of the HPMCAS solution and BD-HPMCAS nanoparticle dispersion were mixed together to produce an oil phase that was 5 mg/mL inverted nanoparticles, 5 mg/mL HPMCAS, and 5 v % methanol in DCM.

The oil phase (500 μL) was added to the bottom of 6 mL of an acidic aqueous phase containing 0.2 wt % poly(vinyl alcohol) (80% hydrolyzed), 140 mM NaCl, and 10 mM HCl in a 20 mL glass scintillation vial. A nanoparticle-in-oil-in-water emulsion was produced by vortex mixing at 1000 rpm for 1 min. After emulsification, the DCM was removed by rotary evaporation at 20° C. and 200 torr for 10 min, followed by 100 torr for 20 min, producing hardened microparticles.

The microparticles were removed from solution by centrifugation and washed three times with water. The microparticles were a dark blue color, indicating high encapsulation efficiency of the BD (FIGS. 2A, 2B). Referring to FIG. 2A, in a vial (200) containing water (205), the microparticles (207) retained the dark blue color. However, referring to FIG. 2B, when dispersed in phosphate buffered saline (PBS) (215) at pH 7, the particles (217) in the vial (210) quickly lost the blue color (1-5 minutes), and the aqueous phase (215) developed a light blue tone indicating that the BD had been released. This was substantiated by optical microscopy, which showed dense microparticles in deionized water, which slowly swelled from the outside-in upon addition of PBS. After 2 minutes, distinct microparticles were no longer visible (FIG. 3). HPMCAS is a pH sensitive polymer and is insoluble in water at low pH and partially soluble at high pH due to the charge on the succinate groups.

### Example 3: Vancomycin Loaded HPMCAS Nanoparticles

Vancomycin was encapsulated in inverted nanoparticles stabilized by HPMCAS 126 using iFNP. The process solvent stream was comprised of DMSO with 10 v % water, 5 mg/mL vancomycin, and 5 mg/mL HPMCAS 126 (Table 4). In Sample 3A, the process solvent contained 60 μg/mL of NaOH (0.5 equivalents with respect to the acid groups on the HPMCAS). In Sample 3B, the process solvent contained 90 μg/mL of NaOH (0.75 equivalents with respect to the acid groups on the HPMCAS). The non-process solvent was DCM.

TABLE 4
The process solvent stream compositions for Example 3. In all cases the solvent was DMSO.
The NaOH equivalents are with respect to the acid groups on the HPMCAS 126.

| | HPMCAS | Vancomycin | | NaOH | |
|---|---|---|---|---|---|
| | Concentration, | Concentration, | Water | Concentration, | NaOH |
| Sample | mg/mL | mg/mL | content | μg/mL | equivalents |
| 3A | 5 | 5 | 10 v % | 60 | 0.5 |
| 3B | 5 | 5 | 10 v % | 90 | 0.75 |
| 3C | 0 | 5 | 10 v % | 0 | n/a |

The process solvent (500 μL) and non-process solvent (500 μL) were rapidly mixed in a CIJ mixer, and the effluent was collected in 4 mL of DCM. Samples 3A and 3B resulted in translucent solutions with no visible aggregates. A control with only vancomycin in the DMSO process solvent stream and no HPMCAS (Sample 3C) resulted in the immediate formation of visible aggregates after mixing with DCM.

The nanoparticle size distributions in Samples 3A and 3B were measured by DLS in DCM, and the PK1 diameter and the polydispersity index PDI from the Malvern Zetasizer DLS software are given in Table 5 and the size distributions are given in FIG. 4. Increasing the amount of NaOH from 0.5 equivalents in Sample 3A to 0.75 equivalents in Sample 3B resulted in a ˜30 nm increase in nanoparticle size.

TABLE 5
Particle diameters and PDIs for Samples 3A and 3B.

| Sample | Diameter, nm | PDI |
|---|---|---|
| 3A | 180 | 0.17 |
| 3B | 210 | 0.17 |

### Example 4: Polymyxin B Loaded HPMCAS Nanoparticles

Polymycin B was encapsulated in inverted nanoparticles stabilized by HPMCAS 126 using iFNP. The process solvent stream was comprised of DMSO with 5 v % water, vancomycin, HPMCAS 126, and NaOH (see Table 6 for the concentrations). Sample 4A was targeting ˜50 wt % polymyxin loading, and Sample 4B was the targeting ˜75 wt % loading. Sample 4C was a control with no HPMCAS stabilizer. For all samples, the non-process solvent was DCM.

TABLE 6
The process solvent stream compositions for Example 4. The NaOH
equivalents are with respect to the acid groups on the HPMCAS 126.

| | HPMCAS | Polymyxin B | NaOH | |
|---|---|---|---|---|
| | Concentration, | Concentration, | Concentration, | NaOH |
| Sample | mg/mL | mg/mL | μg/mL | equivalents |
| 4A | 5 | 5 | 60 | 0.5 |
| 4B | 2.5 | 7.5 | 30 | 0.5 |
| 4C | 0 | 5 | 0 | n/a |

The process solvent (500 μL) and non-process solvent (500 μL) were rapidly mixed in a CIJ mixer, and the effluent was collected in 4 mL of DCM. Samples 4A and 4B resulted in translucent solutions with no visible aggregates. The control with only vancomycin in the DMSO process solvent stream and no HPMCAS (Sample 4C) resulted in the immediate formation of visible aggregates after mixing with DCM.

The nanoparticle size distributions of Samples 4A and 4B were measured by DLS in DCM, and the PK1 diameter and the polydispersity index PDI from the Malvern Zetasizer DLS software are given in Table 7 and the size distributions are given in FIG. 5. Increasing the polymyxin loading from 50 wt % in Sample 4A to 75 wt % in Sample 4B resulted in a ˜30 nm increase in nanoparticle size.

TABLE 7
Particle diameters and PDIs for Samples 4A and 4B.

| Sample | Diameter, nm | PDI |
|---|---|---|
| 4A | 85 | 0.23 |
| 4B | 115 | 0.16 |

### Example 5: Lysozyme Loaded HPMCAS Nanoparticles

Lysozyme was encapsulated in inverted nanoparticles stabilized by HPMCAS 126 using iFNP. The process solvent stream was comprised of DMSO with 10 v % water, lysozyme, HPMCAS 126, and NaOH (see Table 8 for the concentrations). Sample 5A was targeting ˜50 wt % lysozyme loading, and Sample 5B was the targeting ˜75 wt % loading. Sample 5C was a control with no HPMCAS stabilizer. For all samples, the non-process solvent was DCM.

TABLE 8
The process solvent stream compositions for Example 5. The NaOH
equivalents are with respect to the acid groups on the HPMCAS 126.

| | HPMCAS | Vancomycin | NaOH | |
|---|---|---|---|---|
| | Concentration, | Concentration, | Concentration, | NaOH |
| Sample | mg/mL | mg/mL | μg/mL | equivalents |
| 5A | 5 | 5 | 60 | 0.5 |
| 5B | 2.5 | 7.5 | 30 | 0.5 |
| 5C | 0 | 5 | 0 | n/a |

The process solvent (500 μL) and non-process solvent (500 μL) were rapidly mixed in a CIJ mixer, and the effluent was collected in 4 mL of DCM. Samples 5A and 5B resulted in translucent solutions with no visible aggregates. The control with only lysozyme in the DMSO process solvent stream and no HPMCAS (Sample 5C) resulted in the immediate formation of visible aggregates after mixing with DCM.

The nanoparticle size distributions of Samples 5A and 5B were measured by DLS in DCM, and the PK1 diameter and the polydispersity index PDI from the Malvern Zetasizer DLS software are given in Table 9 and the size distributions are given in FIG. 6. Increasing the lysozyme loading from 50 wt % in Sample 5A to 75 wt % in Sample 5B resulted in a ˜100 nm increase in nanoparticle size.

TABLE 9
Particle diameters and PDIs for Samples 5A and 5B.

| Sample | Diameter, nm | PDI |
|---|---|---|
| 5A | 185 | 0.08 |
| 5B | 285 | 0.02 |

### Example 6: Horseradish Peroxidase Loaded HPMCAS Nanoparticles

Horseradish peroxidase (HRP) was encapsulated in inverted nanoparticles stabilized by HPMCAS 126 using iFNP. The process solvent stream was comprised of DMSO with 10 v % water, HRP, HPMCAS 126, and NaOH (see Table 10 for the concentrations). Sample 6A was targeting ˜50 wt % HRP loading, and Sample 6B was the targeting ˜75 wt % loading. Sample 6C was a control with no HPMCAS stabilizer. For all samples, the non-process solvent was DCM.

TABLE 10
The process solvent stream compositions for Example 4. The NaOH
equivalents are with respect to the acid groups on the HPMCAS 126.

| | HPMCAS | Vancomycin | NaOH | |
|---|---|---|---|---|
| | Concentration, | Concentration, | Concentration, | NaOH |
| Sample | mg/mL | mg/mL | μg/mL | equivalents |
| 6A | 5 | 5 | 60 | 0.5 |
| 6B | 2.5 | 7.5 | 30 | 0.5 |
| 6C | 0 | 5 | 0 | n/a |

The process solvent (500 μL) and non-process solvent (500 μL) were rapidly mixed in a CIJ mixer, and the effluent was collected in 4 mL of DCM. Samples 6A and 6B resulted in translucent solutions with no visible aggregates. The control with only HRP in the DMSO process solvent stream and no HPMCAS (Sample 6C) resulted in the immediate formation of visible aggregates after mixing with DCM.

The nanoparticle size distributions of Samples 6A and 6B were measured by DLS in DCM, and the PK1 diameter and the polydispersity index PDI from the Malvern Zetasizer DLS software are given in Table 11 and the size distributions are given in FIG. 7. Increasing the HRP loading from 50 wt % in Sample 6A to 75 wt % in Sample 6B resulted in a ˜45 nm increase in nanoparticle size.

TABLE 11
Particle diameters and PDIs for Samples 6A and 6B.

| Sample | Diameter, nm | PDI |
|---|---|---|
| 6A | 120 | 0.08 |
| 6B | 165 | 0.05 |

### Example 7: Maltodextrin-Core Nanoparticles Stabilized by Eudragit L100-55

In order to test the ability of a methacrylate-based random copolymer to stabilize a hydrophilic compound in iFNP, formulations using maltodextrin (dextrose equivalents 4-7) and Eudragit L100-55 were prepared. The solvent stream was dimethyl sulfoxide (DMSO) with 5 mg/ml maltodextrin and 5 mg/ml Eudragit polymer. This corresponds to 50% loading of the model biologic. 5 vol % deionized water was added to this stream. The antisolvent stream was an equal volume stream of chloroform, which contained (when noted) 10 vol % of a 0.139M solution of CaCl₂ in methanol. The quench was 9 volumes of chloroform. In some cases, ammonia was added to strengthen the calcium interactions with acidic monomers in the L100-55 polymer Ammonia was added with vigorous stirring as a solution in methanol (50 μL of 0.139M solution, NH₃ basis; this solution was formed by diluting ammonium hydroxide in methanol to the target concentration). If no calcium was included, ammonia in methanol (0.139M NH₃ basis) was added directly to the antisolvent stream at 10 vol %. The nanoparticles were prepared by iFNP using a CIJ mixer. The nanoparticles were characterized by dynamic light scattering using a Malvern Zetasizer ZS. Intensity distributions are reported when particle size distributions are shown. Peak sizes are determined from a CONTIN analysis of the correlation function that is implemented by the Malvern software.

The control formulation without polymer stabilizer in the DMSO stream was polydisperse and consisted of large nanoparticles or micron-scale aggregates upon visual inspection. Formulations with the polymer stabilizer formed stable nanoparticles with narrow size distributions as measured by the polydispersity index (PDI) generated by the Malvern software from a cumulants analysis (PDI values of less than 0.2). The inclusion of ammonia in the antisolvent stream reduced nanoparticle size. The presence of CaCl₂ in the antisolvent stream also resulted in uniform nanoparticle size distributions. These results are summarized in Table 12. Ammonia was added at 0.5 equivalents, assuming a molecular weight of 32,000 g/mol and 50% acidic monomer content. The calcium was added at a 1:1 charge equivalency with the acid groups using the same monomer content assumption.

To probe particle stability, 13 wt % NaCl in water solutions were prepared for use in an extraction that removes DMSO and dissolves unprotected maltodextrin. The solution was treated to make it either acidic (0.1M acetic acid or citric acid) or basic (pH 9 by NaOH adjustment). Samples were extracted for 30 min to 1 hour on a rotary wheel. The control saw steep drops in light scattering signal. The copolymer-stabilized particles were stable to the acidic brine solution extraction as would be expected for the Eudragit solubility profile. The presence of Ca²⁺ with or without NH₃ resulted in minimal size changes (5-7%) during the acidic extraction (Table 12, FIGS. 8A, 8B). These data demonstrate the successful stabilization of hydrophilic-core nanoparticles by a methacrylate-based random copolymer.

TABLE 12
Dynamic Light Scattering analysis of maltodextrin-core nanoparticles
stabilized by Eudragit L100-55 with different additives. The
nanoparticles were stable to extraction for the removal of DMSO.

| | Inverse NP | | After Acid Extraction | |
|---|---|---|---|---|
| | size (nm) | PDI | Size (nm) | PDI |
| Maltodextrin (MD) | 631 | 0.526 | — | — |
| MD + L100-55 | 265 | 0.027 | 279 | 0.062 |
| +NH₃ | 113 | 0.163 | 261 | 0.161 |
| +CaCl₂ | 166 | 0.055 | 174 | 0.084 |
| +NH₃/CaCl₂ | 187 | 0.037 | 201 | 0.069 |

### Example 8: Parameter Variation in Maltodextrin Nanoparticle Formulation

To evaluate the design space of the inverse nanoparticle formation process by iFNP, the maltodextrin loading, crosslinker identity, and ammonia equivalents were systematically varied. Mechanistically, it would be expected to find a window in the substoichiometric quantity of ammonia where sufficient base has been added to promote crosslinking. Excess base is generally undesirable because of potential degradation reactions to encapsulated biologics. Ammonia was added at quantities that remained substoichiometric given the assumptions detailed in Example 7. Without detailed knowledge of the acid monomer content, the appropriate amount of base to add may be best found empirically.

Ammonia was incorporated into the antisolvent stream as described in Example 7 and maltodextrin nanoparticles were prepared by iFNP. The particles were generally stable to acid extraction. Higher base equivalents resulted in higher PDIs for the initial particles. The largest size changes during extraction occurred at 0.25 eq and 0.5 eq, though the particles remained stable. The data are summarized in Table 13.

TABLE 13
Dynamic Light Scattering analysis of the effect of ammonia equivalents
on nanoparticles of maltodextrin stabilized by Eudragit L100-55. The
particles were stable to acid extraction to remove DMSO.

| | Inverse NP | | After Acid Extraction | |
|---|---|---|---|---|
| NH₃ (Eq) | Size (nm) | PDI | Size (nm) | PDI |
| 0 eq | 229 | 0.11 | 232 | 0.10 |
| 0.1 eq | 146 | 0.124 | 136 | 0.02 |
| 0.25 eq | 119 | 0.136 | 204 | 0.02 |
| 0.5 eq | 113 | 0.163 | 261 | 0.161 |
| 0.75 eq | 141 | 0.2 | 170 | 0.044 |

The effect of maltodextrin loading was evaluated at a constant total mass concentration of 10 mg/ml in DMSO. The relative fraction of maltodextrin was varied and the resulting nanoparticles were characterized by DLS. The size was relatively invariant but PDI was larger for lower loading (Table 14).

TABLE 14
The effect of maltodextrin loading (mass basis) on
nanoparticle size with an Eudragit L100-55 stabilizer as
assessed by Dynamic Light Scattering analysis.

| MD Loading (%) | size (nm) | PDI |
|---|---|---|
| 16.8 | 198 | 0.35 |
| 25 | 280 | 0.19 |
| 50 | 201 | 0.17 |
| 75 | 200 | 0.11 |

The crosslinker effect was studied by replacing the CaCl₂ solution in the antisolvent stream with an FeCl₃ solution. The molarity of this methanol solution was adjusted to maintain a 1:1 charge ratio with the putative composition of acid monomer units. The particles were bimodal and larger, but were still nanometer-scale (FIG. 9). The Fe-crosslinked particles were stable to swelling in DMSO but aggregated some during the acidic extraction while the Ca-crosslinked particles did not exhibit visible aggregation.

### Example 9: Additional Eudragit Stabilizers are Effective in iFNP

Maltodextrin nanoparticles were made by iFNP with a range of Eudragit stabilizers. The nanoparticles were prepared as in Example 7 at 50% loading with and without CaCl₂. No ammonia addition was carried out. The particles were extracted with a 13 wt % NaCl in water solution that contained 0.1M citric acid to remove DMSO. The particle behavior was characterized by DLS.

The results are summarized in Tables 15 and 16 and indicate two important points. First, all random copolymers tested were found to successfully stabilize inverse nanoparticles of maltodextrin. The addition of Ca²⁺ was important for stability in the acid extraction step. Without the crosslinker, particle aggregation was extensive (visual observation). The presence of the crosslinker ensured that only minor swelling occurred during the extraction step. Both pH-responsive and pH-independent Eudragit polymers were successfully used to stabilize the iFNP-produced nanoparticles.

TABLE 15
The effect of Eudragit stabilizer on the particle characteristics assessed Dynamic
Light Scattering analysis for maltodextrin encapsulation. The particles were
assembled with a Ca²⁺ counterion present in the antisolvent stream.

| With Ca²⁺ | Inverse NP | | After Extraction | |
|---|---|---|---|---|
| Sample Name | Size (nm) | PdI | Size (nm) | PdI |
| L100-55 | 216 | 0.025 | 209 | 0.051 |
| L100 | 290 | 0.125 | 363 | 0.126 |
| S100 | 153 | 0.11 | 156 | 0.12 |
| RS-PO | 155 | 0.061 | 179 | 0.081 |
| RL-PO | 146 | 0.062 | 181 | 0.101 |

TABLE 16
The effect of Eudragit stabilizer on the particle characteristics assessed Dynamic
Light Scattering analysis for maltodextrin encapsulation. The particles were
assembled without a Ca²⁺ counterion present in the antisolvent stream.

| Without Ca²⁺ | Inverse NP | | After Extraction | |
|---|---|---|---|---|
| Sample Name | Size (nm) | PdI | Size (nm) | PdI |
| L10055 | 408 | 0.204 | Aggregation | |
| L100 | 638 | 0.32 | Aggregation | |
| S100 | 293 | 0.086 | 718 | 0.312 |
| RS-PO | 143 | 0.16 | 184 | 0.229 |
| RL-PO | 58 | 0.275 | 115 | 0.279 |

| | | | |
|---|---|---|---|
| E-PO | 144 | 0.163 | Aggregation |

### Example 10: Solvent Exchange into THF for Microparticle Spray Drying

In order to test the particle suitability for use in Processing Route 3 (described above), the maltodextrin nanoparticles were processed through the extraction and then a solvent swap into THF. The particles were made as in Example 7 using L100-55 or S100. The acid extraction was carried out for 1 hour. Then 6 ml (1.2 volumes) of THF was added and the mixture was concentrated using a rotovap to 1 ml (0.2 volumes). This was repeated twice more to afford a colloidal dispersion in THF. Particle stability was evaluated visually and by DLS.

Both L100-55 and 5100 stabilized particles successfully throughout the solvent swap process as shown in Table 17. It is clear that the swap process or exposure to THF resulted in some particle swelling, about 50 nm in both cases. However, this did not come at the expense of particle PDI, which increased only slightly during processing. Produced in sufficient quantities, these THF dispersions could be spray dried to afford a powder which could then be formulated for oral delivery. Additional excipients could be included in that THF stream or included during powder formulation later. It is also feasible that the spray drying could be done for inverse nanoparticles in chloroform or DCM.

TABLE 17
Stability of Eudragit-stabilized nanoparticles of maltodextrin to
process steps. The nanoparticles were characterized by Dynamic
Light Scattering after formation and then again after extraction
and solvent exchange by rotovap into tetrahydrofuran.

| | Inverse NP | | Post Swap | |
|---|---|---|---|---|
| | Size (nm) | PDI | Size (nm) | PDI |
| L100-55 | 203 | 0.03 | 272 | 0.07 |
| S100 | 153 | 0.06 | 200 | 0.10 |

### Example 11: Extension to Representative Model Biologics of Varying Properties

To form horseradish peroxidase (HRP) nanoparticles stabilized by L100-55 Eudragit, the basic process from Example 7 was employed. Horseradish Peroxidase (Type I from Millipore Sigma) was dissolved in DMSO at 10 mg/ml without further purification. Eudragit L100-55 was dissolved in DMSO at 12.5 mg/ml. A DMSO solution containing 7 mg/ml of L100-55 and 3 mg/ml of HRP with 5 vol % deionized water was prepared. The chloroform antisolvent contained Ca(NO₃)₂ at 4.6 mg/ml and 10 vol % methanol. Mixing was carried out according to the iFNP process in a confined impinging jet mixer at flow rates of greater than 60 ml/min per stream. The mixed streams were collected in a quench bath of chloroform such that the final DMSO content was 10%. Vancomycin nanoparticles were prepared similarly except that the DMSO stream contained 5 mg/ml of vancomycin and the Eudragit copolymer. Deionized water content was also adjusted to 10 vol % in the stream. The formulation with L100 did not contain the calcium salt.

Solutions were visually inspected for aggregates and analyzed by dynamic light scattering as described in example 7. Controls without Eudragit stabilizer formed visible aggregates. The DLS characterization results are summarized in Table 18 and depicted in FIGS. 10 and 11. All formed stable nanoparticles. The vancomycin samples were all narrow size distributions.

TABLE 18
The effect of Eudragit stabilizer on the particle characteristics assessed
by Dynamic Light Scattering analysis for different encapsulated biologics.
The particles were assembled with a Ca²⁺ counterion present in the
antisolvent stream with the exception of Eudragit L100.

| Biologic | Eudragit | Loading | Size (nm) | PDI |
|---|---|---|---|---|
| HRP | L100-55 | 30% | 206 | 0.24 |
| Vancomycin | L100-55 | 50% | 186 | 0.13 |
| Vancomycin | RL | 50% | 124 | 0.13 |
| Vancomycin | S100 | 50% | 379 | 0.15 |
| Vancomycin | RS | 50% | 147 | 0.11 |
| Vancomycin | L100 | 50% | 539 | 0.09 |

What is claimed:

1. A method for encapsulating a water-soluble agent, comprising the steps of:
dissolving a water-soluble agent and a stabilizing random copolymer having hydrophilic and hydrophobic groups in a process solvent having a first polarity to form a process stream;
mixing the process stream with a non-process solvent stream, the non-process solvent stream having a polarity less than the first polarity; and
forming an inverse nanoparticle dispersion comprising a plurality of nanoparticles, by precipitating the water-soluble agent in the non-process stream and allowing the stabilizing random copolymer to organize such that the water-soluble agent and the hydrophilic group of the stabilizing random copolymer form a core of each nanoparticle while the hydrophobic group of the stabilizing random copolymer forms a stabilizing shell of each nanoparticle.

2. The method according to claim 1, wherein the water-soluble agent is selected from the group consisting of an amino acid, a peptide, a protein, an antibody, DNA, RNA, a saccharide, a lysozyme, a small molecule therapeutic, tobramycin, vancomycin, and an imaging agent.

3. The method according to claim 1, wherein the non-process solvent is selected from the group consisting of chloroform, dichloromethane (DCM), an alkane, hexane, an ether, diethyl ether, tetrahydrofuran (THF), toluene, acetone, or combinations. For example, the non-process solvent can be chloroform, dichloromethane, acetone, or combinations thereof.

4. The method according to claim 1, wherein a first nanoparticle is crosslinked to a second nanoparticle.

5. The method according to claim 1, further comprising coating the nanoparticles.

6. The method according to claim 1, further comprising aggregating the nanoparticles into microparticles.

7. The method of claim 6, wherein the inverse nanoparticles are aggregated by atomizing the inverse nanoparticle dispersion and drying the inverse nanoparticle dispersion during a spray drying step to form solid, essentially dry microparticles.

8. A microparticle, comprising
an aggregation of inverse nanoparticles,
wherein each inverse nanoparticle comprises a core comprising a hydrophilic group of a stabilizing random copolymer and a shell comprising a hydrophobic group of the stabilizing random copolymer, and
wherein a water-soluble agent is within the core.
